(19) United States
(12) Patent Application Publication
(10) Pub. No.: US 2024/0252418 A1
(43) Pub. Date: Aug. 1, 2024

(54) Additives (Adjuvants, Anti Freezing Agents, Antimicrobial and Antioxidants) in Natural Deep Eutectic Solvents and Mixtures

(71) Applicant: Bioeutectics Corporation, Wilmington, DE (US)

(72) Inventors: Tomás Silicaro, Nordelta (AR); Sergio David Pasini Cabello, Ayacucho (AR); Maria Romina Canales, Junin mendoza (AR)

(73) Assignee: Bioeutectics Corporation, Wilmington, DE (US)

(21) Appl. No.: 18/427,860

(22) Filed: Jan. 31, 2024

Related U.S. Application Data
(60) Provisional application No. 63/442,122, filed on Jan. 31, 2023.

(51) Int. Cl. A61K 8/86; A61K 8/04; A61K 8/34; A61Q 17/00

(52) U.S. Cl. CPC A61K 8/86; A61K 8/04; A61K 8/345; A61Q 17/005

(57) ABSTRACT

NADES (natural deep eutectic solvents) are disclosed that can be used as additives to give products unexpectedly superior properties or synergistic properties relative to the use of the product alone. The NADES of the present invention as disclosed herein demonstrate superior properties as additives to other products as adjuvants, as anti-freeze agents, as anti-microbial agents and/or as additives that are added to personal care products.

### CROSS-REFERENCE TO RELATED APPLICATIONS

The present application claims priority under 35 USC 119(e) to U.S. Provisional Application No. 63/442,122 filed Jan. 31, 2023, the entire contents of which is incorporated by reference.

### FIELD OF THE INVENTION

The present invention relates to using eutectic solvent systems, NADES (natural deep eutectic solvents) as additives to give products unexpectedly superior properties or synergistic properties relative to the use of the product alone. The NADES of the present invention as disclosed herein demonstrate superior properties as additives to other products as adjuvants, as anti-freeze agents, anti-microbial agents and/or as additives that are added to personal care products.

### BACKGROUND OF THE INVENTION

Deep eutectic solvents or DESs are solutions of Lewis or Brønsted acids and bases which form a eutectic mixture. Deep eutectic solvents are highly tunable by varying the structure of the components or by varying the relative ratios of various components in the mixture. Because these are complicated systems that have widely varying properties, they have a wide variety of potential applications, including their use in catalysis, separation techniques, and electrochemical processes. The parent components of deep eutectic solvents tend to engage in complex hydrogen bonding networks, which means that the mixture tends to have significant freezing point depressions relative to the parent compounds/components in the mixture. Sometimes the individual components in the mixture may be solids at room temperature and atmospheric pressure, but when they are mixed together at room temperature and atmospheric pressure, the mixture may be a liquid that has a severely depressed freezing point (e.g., 10° C.).

The term “eutectic” was first coined in 1884 by British chemist and physicist Frederick Guthrie. The first generation of eutectic solvents were based on mixtures of quaternary ammonium salts with hydrogen bond donors such as amines and/or carboxylic acids. Natural deep eutectic solvents (NADES) are biologically based deep eutectic solvents which are composed of two or more compounds that are generally plant based primary metabolites, i.e., organic acids, sugars, alcohols, amines and amino acids. Water may also be present as part of the solvent, as water is sometimes difficult to remove due to its inability to be easily evaporated.

Much of the study of eutectic solvents since Frederick Guthrie coined the term “eutectic” has involved solvent mixtures wherein at least one of the components is a metal based solvent. However, the discharge of metals from these solvent systems has demonstrated many of the drawbacks associated with metal leaching, and its associated health, environmental, and safety related issues. Accordingly, there has been some recent interest in non-metal containing eutectic systems.

U.S. patent Ser. No. 10/865,334 relates to a process for extracting materials from biological material, which process is characterized in that the naturally occurring biological material is treated with an extract consisting of a deep eutectic solvent of natural origin or an ionic liquid of natural origin to produce a biological extract of natural origin dissolved in the solvent or ionic liquid.

IN202041012054A relates to a synergistic formulation that can be adopted as a medium for easy extraction of phytochemicals from natural sources—biomass and herbs. The medium is adopted in the preparation of feed supplements for livestock, enriched in nutritional value.

U.S. Pat. No. 10,981,084B2 relates to the use of coconut water as an extraction solvent, to extraction methods using coconut water and extracts obtained by extraction with coconut water. The discussion of deep eutectic solvents appears in the background of the invention.

WO2022101490A1 relates to the development of Natural Deep Eutectic Solvents (NADES) using natural products, like sugars, organic bases and organic acids, as starting compounds. These solvents can be used for the extraction of bioactive compounds from natural sources, such as cork; agricultural wastes, including grape seed and peels; tomato; olive oil; and plants (teas, eucalyptus, lavender, or others), and from fish skin and bones. The extractives may then be further formulated with active topical cosmetic components to prepare cosmetic compositions. WO2022101490A1 focuses on the application of NADES for the extraction of chemical compounds from natural sources. The extraction methods applied use the “enfleurage” method, which is an ultrasound-assisted extraction and sealed system extraction. The natural extracts isolated and obtained can then be applied directly in cosmetic formulations without further purification.

EP3971230A1 relates to a deep eutectic solvent (DES) comprising at least one carboxylic acid which comprises at least two carboxylic acid functional groups with a number of carbon atoms that range from 4 to 10; at least one alcohol which comprises at least two alcohol functional groups, with the alcohols having 2 to 12 carbon atoms, polyethylene glycol and polypropylene glycol; and water in an amount of from 10 to 50 wt. % of the total weight of the deep eutectic solvent. This reference describes the use of a DES as a solvent system for solubilizing lignin from a lignin containing material, or the use of the DES for preparing a lignin-prepolymer which can be subsequently used in applications such as for producing films, coatings, insulating foams, adhesives, binders, composites or for fibre sizing or for radical curing.

EP3693418A1 relates to a solvent composition, in particular a solvent composition with components of natural, non-petrochemical origin. It relates to a solvent composition based on compounds of vegetable origin deriving from the fermentation of carbohydrates, wherein those carbohydrates are glucose, fructose, sucrose, starches, cellulose and mixtures thereof. Although this reference discloses solvent mixtures, it does not appear to relate to deep eutectic solvents.

EP4011353 relates to eutectic solvents formed from the mixture of ascorbic acid (vitamin C) in combination with Betaine and a third component selected from the group comprising Water, Ethanol, Glycerol, Diols and/or Triols with 6 or less than 6 C-atoms, especially 1,3-Propanediol, Butylene Glycol and Hexanediol. The eutectic solvents allow for incorporation of these active ingredients into cosmetic compositions.

Although the above cited references do tangentially relate to eutectic mixtures and some relate to the isolation/extraction of mixtures, none or the references disclose or suggest the use or addition of NADES in the ratios described herein with additives to systems that can be used for a plurality of purposes with surprisingly superior properties relative to mixtures that are currently on the market. By using different components or different ratios of these eutectic solvent mixtures with additives that differ from the prior art, the present invention is able to attain new and/or surprisingly superior properties.

### BRIEF SUMMARY OF THE INVENTION

In an embodiment, the present invention relates to systems, compositions, and methods of using the systems and compositions which comprise NADES (natural deep eutectic solvent) mixtures of solvents as additives. When the eutectic mixtures are used as additives with products that are used for certain purposes, the addition of the eutectic mixture often provides superior and/or synergistic results relative to the use of the product used alone.

### BRIEF DESCRIPTION OF THE SEVERAL VIEWS OF THE DRAWING

FIG. 1 shows a comparison of the wetted area of TX (Triton X-100) and various eutectic products (NADES) on a paper sheet.

FIG. 2 shows five graphs with each graph representing different compositions of NADES that can be used as an additive that improves the performance of adjuvants.

FIG. 3 shows the results in test tubes of various samples.

FIG. 4 shows a bar graph showing the relative wetting area of different solvent systems.

FIG. 5 shows a solubility test at the working concentration of 14% w/w analyte/solvent, initially in traditional solvents and in the invention solvents, at 30° C. where 1, and 0 are taken for analytes that are soluble or insoluble, respectively.

FIG. 6 shows IR spectra of NaAcGlH at various temperatures.

FIG. 7 shows IR spectra of LGH at various temperatures.

FIG. 8 shows a viscosity curve of pure LGH.

FIG. 9 shows the viscosity curve of LGH containing 30% water.

FIG. 10 shows a viscosity curve of NaAcGlH for the pure solvent system.

FIG. 11 shows a viscosity curve of NaAcGlH that contains 50% water.

### DETAILED DESCRIPTION OF THE INVENTION

In an embodiment, the present invention relates to the use of the eutectic mixtures of solvents as additives. When the eutectic mixtures are used as additives with products that are used for certain purposes, the addition of the eutectic mixture often provides superior and/or synergistic results relative to the product used alone.

In an embodiment, the present invention relates to adding eutectic mixtures as additives to different products such as personal care products, to antifreezes, and/or to anti-microbial products (or compositions). In an embodiment, the addition of eutectic mixtures of solvents makes the personal care products, antifreezes, and/or anti-microbial products better.

The following examples are given to illustrate various embodiments of the present invention. They should, however, not be construed to limit the invention.

### Examples

### Adjuvants

### Laboratory Tests

### Product Preparation

Several eutectic systems were prepared by heating the combination of eutectic compounds on a magnetic stirrer until a clear liquid formed. The temperature and time to generate the clear eutectic solutions were optimized in each case. Both hydrophilic and hydrophobic eutectic systems were prepared. The hydrophilic eutectics system used was UL, which is composed of Urea (20-40%), Lactic Acid (60-80%). The hydrophobic eutectics systems prepared included MT, comprised of Menthol (20-80%), Thymol (20-80%), MOA, comprised of Menthol (10-70%), Oleic Acid (30-90%), and BOA, comprised of Borneol (10-30%), Oleic Acid (60-90%).

### Wetted Area Effects (Pure Systems) Tests

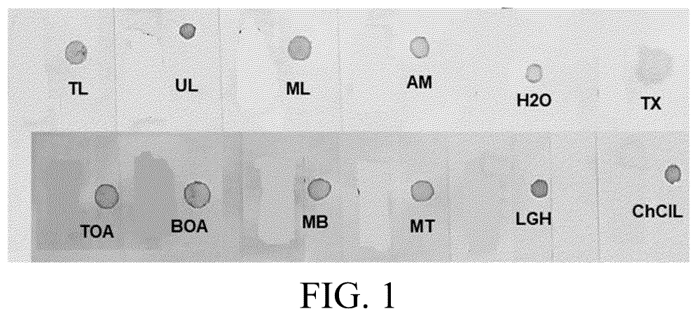

A general procedure was developed to evaluate the wetted area of droplets. The test system was prepared with 1% of the eutectic products in water, and a miscible dye, which allowed the visualization of the wetted area droplets. To compare the ability of the pure systems to a known adjuvant, TX (Triton X-100), a nonionic surfactant that has a hydrophilic polyethylene oxide chain with, on average, 9.5 ethylene oxide units) was used and was prepared under the same conditions and compared with a water/dye blank. Next, 5 μL of each system to be compared was placed on a paper sheet, as shown in FIG. 1.

FIG. 1 shows a comparison of the wetted area of the various pure products on a paper sheet. TX represents Triton X-100, the various wetted drops that start with HF represent hydrophilic products, and the various wetted drops that start with HB represent the hydrophobic products.

### Comparison on a Paper Sheet

The wetted drops of TX, water, and the various hydrophilic and hydrophobic eutectic products of the present invention were measured quantitatively and those measurements are shown in table 1.

TABLE 1

| | | Relative size (%) | Relative size (%) |
|---|---|---|---|
| | Area | with reference to | with reference to |
| 5 μL (1%) | (mm²) | TX | H₂O |
| TX | 23.98 | 100.00 | 424.82 |
| H₂O | 5.64 | 23.54 | 100.00 |
| UL | 4.90 | 20.45 | 86.86 |
| MT | 9.29 | 38.75 | 164.60 |
| MOA | 9.72 | 40.55 | 172.26 |
| BOA | 11.87 | 49.48 | 210.22 |

From Table 1, it should be apparent that the tested systems' wetted areas (mm²) showed that TX by itself demonstrated the best results relative to the other systems under study. The products of the present invention (i.e UL, MT, MOA, and BOA) showed wetted drops that were about 50% or less of the wetted area surface relative to the performance of TX.

### Comparison in Waxy Leaves

In a subsequent experiment, the wetted area of droplets was tested on waxy leaves of Epipremnum aureum. The quantitative results of those tests appear in Table 2. In this test, the products of the present invention demonstrated an efficiency of around 70% or less relative to the blank (i.e., the performance is relative to Triton-X at the same concentration as the tested NADES). In the case of UL, the wetted area of the drop did not increase, but there was a permeation observed both in the paper sheet as well as on the leaf of the plant.

TABLE 2
Data of wetted area on waxy leaves.

| | Relative size (%) with | Relative size (%) with |
|---|---|---|
| | reference to TX | reference to H₂O |
| TX | 100.00 | 333.18 |
| H₂O | 30.01 | 100.00 |
| UL | 8.20 | 27.32 |
| MT | 36.86 | 122.80 |
| MOA | 69.69 | 232.18 |
| BOA | 56.65 | 188.75 |

### Wetted Area Effects (Mixture Systems)

With the results from the pure systems on paper and a waxy leaf shown in tables 1 and 2, a subsequent test was performed wherein mixtures of the products of the present invention with TX were compared on a paper sheet. Mixtures comprising TX and the products under study were evaluated at 0.9, 0.75, 0.50, 0.25, and 0.10% (1% total concentration). In these mixtures, significant improvements were observed in the wetted area of drops relative to TX alone. Table 3 shows the quantitative results of these tests. Compared to TX/water, the mixture with TX/BOA (50/50%) increased the wetted area by around 190%, systems with TX/BOA and TX/BOA (75/25%) increased the wetted area by around 180%, while TX/BOA (25/75%) increased the wetted area by around 150% (see Table 3).

TABLE 3
Comparison of the wetting area on a paper
sheet (with the mixture systems)

| | | | Relative size (%) |
|---|---|---|---|
| | | Area | with reference to |
| Component A % | Component B % | (mm²) | TX/H₂O |
| TX 0.1 | H₂O 0.9 | 18.05 | 100.00 |
| TX 0.1 | UL 0.9 | 15.65 | 86.69 |
| TX 0.1 | MT 0.9 | 12.44 | 68.93 |
| TX 0.1 | TOA 0.9 | 11.64 | 64.50 |
| TX 0.1 | BOA 0.9 | 17.84 | 98.82 |
| TX 0.25 | H₂O 0.75 | 18.99 | 100.00 |
| TX 0.25 | UL 0.75 | 15.38 | 81.01 |
| TX 0.25 | MT 0.75 | 20.27 | 106.75 |
| TX 0.25 | TOA 0.75 | 20.91 | 110.13 |
| TX 0.25 | BOA 0.75 | 28.38 | 149.51 |
| TX 0.5 | H₂O 0.5 | 17.78 | 100.00 |
| TX 0.5 | UL 0.5 | 13.06 | 73.42 |
| TX 0.5 | MT 0.5 | 26.38 | 148.35 |
| TX 0.5 | TOA 0.5 | 22.38 | 125.83 |
| TX 0.5 | BOA 0.5 | 34.21 | 192.34 |
| TX 0.75 | H₂O 0.25 | 17.84 | 100.00 |
| TX 0.75 | UL 0.25 | 27.21 | 152.54 |
| TX 0.75 | MT 0.25 | 33.35 | 186.98 |
| TX 0.75 | TOA 0.25 | 27.21 | 152.54 |
| TX 0.75 | BOA 0.25 | 30.31 | 169.91 |
| TX 0.9 | H₂O 0.1 | 15.81 | 100.00 |
| TX 0.9 | UL 0.1 | 12.60 | 79.73 |
| TX 0.9 | MT 0.1 | 22.88 | 144.76 |
| TX 0.9 | TOA 0.1 | 19.79 | 125.17 |
| TX 0.9 | BOA 0.1 | 17.09 | 108.11 |

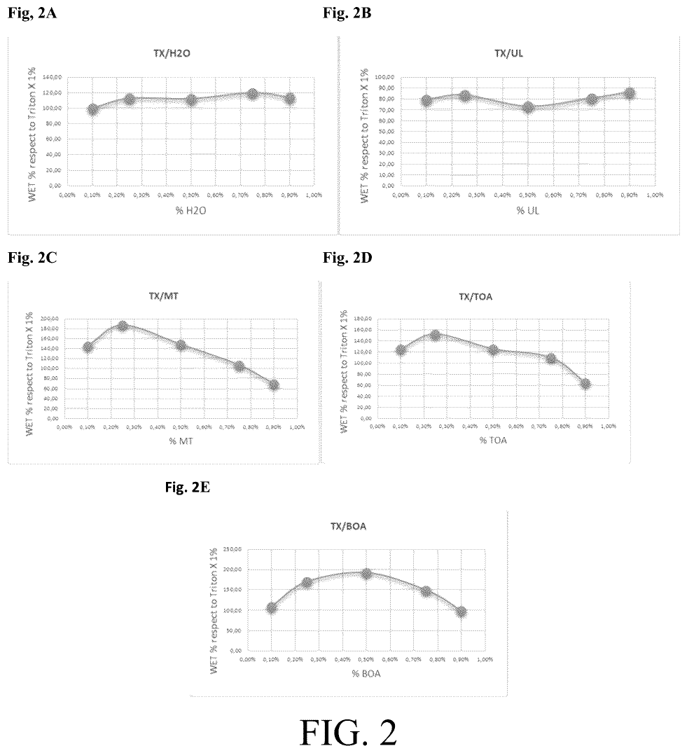

FIG. 2 shows five graphs with each graph representing the different compositions of the wetted area in mixture systems. For example, graph FIG. 2A shows the graph that contains TX and water, FIG. 2B shows a graph that contains TX and UL, FIG. 2C shows a graph that contains TX and MT, FIG. 2D shows a graph that contains TX and TOA, and FIG. 2E shows a graph that contains TX and BOA. Thus, it is apparent that the addition of the eutectic mixtures of the present invention in the relative correct amounts and the correct compositions had a synergistic effect on the size of the wetted drop relative to the system that contained TX and water.

### Evaporation Time Measurements

Evaporation time was analyzed after applying 5 μL (one drop) of a 1% solution in water, on a glass slide at room temperature. The results of the experiments are shown in Table 4, and the results show that the evaporation time is similar in all cases so there does not seem to be any apparent advantage of one system relative to the other for this test. Also in most cases, after the drop evaporated, a very thin film remains that represents the wetted effect.

| | Evaporation | The relative evaporation time |
|---|---|---|
| (1%) in H2O | time (min) | with reference to TX (min) |
| TX | 19.73 | 1.00 |
| UL | 12.73 | 0.65 |
| MT | 19.98 | 1.01 |
| MOA | 16.83 | 0.85 |
| TOA | 18.68 | 0.95 |

### Methylated Seed Oil (MSO)

Market trends will continue to move towards more selective, less toxic, less persistent, and quickly biodegradable pesticides. Adjuvants should be considered as management tools that can improve not only the performance level of pesticides but also the consistency of results. Adjuvants improve or facilitate the management of the physical characteristics of pesticides and thus their action by reducing and minimizing losses, maximizing the effect of the products used. The properties of an adjuvant determine its functionality and these in turn are determined by the design and characteristics of the formulation. Functionality specifically responds to the chemistry, the proportion of the components, and the dose (the amount used per area).

New pesticides tend to use more active molecules, produced in highly concentrated formulations, which are more expensive and require much lower doses than conventional pesticides. In this regard, adjuvants can contribute to effective application (droplet coverage, wetting, deposition, retention; penetration, and translocation) to reduce the margin of error.

The use of adjuvants offers considerable economic and environmental benefits due to the possibility of maximizing the action of the active ingredients. However, it is fundamental to employ adjuvants from renewable sources and not genetically modified organisms (GMOs). In this context, Bioeutectics can be an excellent supplier for this kind of compound. Several natural compounds can be employed as adjuvants working like surfactants/emulsifiers or as humectants replacing actual petrochemical adjuvants.

Therefore, this invention can be a real replacement for MSO adjuvant.

### Laboratory Tests

Several solvents were mixed with silicone surfactant Silwet 641, to evaluate their miscibility. The selection criteria was the miscibility of the proposed solvents with Silwet after 1 minute of shaking. The tests were performed in a mixture of 20% Silwet 641 and 80% of the proposed solvent, respecting the proportions of the data sheet for this formulation.

After that, mixes previously formed were evaluated for emulsification capability. To do this, mixes were added to water in a dosage of 1%, based on the datasheet for the commercial formulation. Stability was evaluated at 30 minutes, 2 hours, and 24 hours (see Table XX). 15 products remained stable after 24 hours. The composition of these solvents is as follows: AT: Camphor (30-60%), Thymol (40-70%); MBA: Menthol (60-80%), Borneol (10-30%), Camphor (10-30%); TOA: Thymol (10-45%), oleic acid (50-90%); GeOA: Geraniol (10-40%), Oleic Acid (30-90%) MOA: Menthol (10-70%), Oleic Acid (30-90%); EucCy: Eucalyptol (10-80%), Cymene (20-90%); EucA: Eucalyptol (10-80%), Camphor (20-90%); EucOA: Eucalyptol (20-80%), Oleic Acid (30-80%); OALi: Oleic Acid (30-80%), Limonene (20-90%); OACy: Oleic Acid (10-80%), Cymene (30-70%); OAPi: Oleic Acid (20-80%), Pinene (10-40%); GlBM: Glycerol (20-60%), Menthol (10-50%), Bomeol (10-40%); MLuA: Menthol (10-60%), Lauric Acid (20-50%); EugEucT: Eugenol (10-80%), Eucalyptol (10-90%), Thymol (20-80%); OctDecLuA: Octanoic Acid (10-40%), Decanoic Acid (10-60%), Lauric Acid (5-70%).

Next, these 15 solvents were used as the basis for the next trial. A wetting area study was carried out. This was done by adding 5 μL of dye to 1000 μL of the surfactant/product mix and placing a 5 μL drop of this mixture on a piece of paper. The wetting area was then determined computationally using ImageJ and normalized taking as 100% of the wetting area of the commercial adjuvant. Both images and a summarized table are presented in the following Table 5.

TABLE 5

| | | | Stability | | |
|---|---|---|---|---|---|
| List | Wetting Area | | | | 24 h |
| Code | mm² | % | 2 to 5° C. | 54° C. | emulsified |
| MSO | 686 | 100 | Yes | Yes | Yes |
| AT | 796 | 116.03 | Yes | Yes | Yes |
| MBA | 436.5 | 63.63 | Yes | Yes | Yes |
| TOA | 634 | 92.42 | Yes | Yes | Yes |
| GeOA | 674.5 | 98.32 | Yes | Yes | Yes |
| MOA | 331.5 | 48.32 | Yes | Yes | Yes |
| EucCy | 1303 | 189.94 | Yes | Yes | Yes |
| EucA | 707.5 | 103.13 | Yes | Yes | Yes |
| EucOA | 1036.5 | 151.09 | Yes | Yes | Yes |
| OALi | 723 | 105.39 | Yes | Yes | Yes |
| OACy | 461.5 | 67.27 | Yes | Yes | Yes |
| OAPi | 550 | 80.17 | Yes | Yes | Yes |
| GlBM | 578 | 84.26 | Yes | Yes | Yes |
| MLuA | 501 | 73.03 | Yes | Yes | Yes |
| EugEucT | 377 | 54.96 | Yes | Yes | Yes |
| OctDecLuA | 654 | 95.34 | Yes | Yes | Yes |

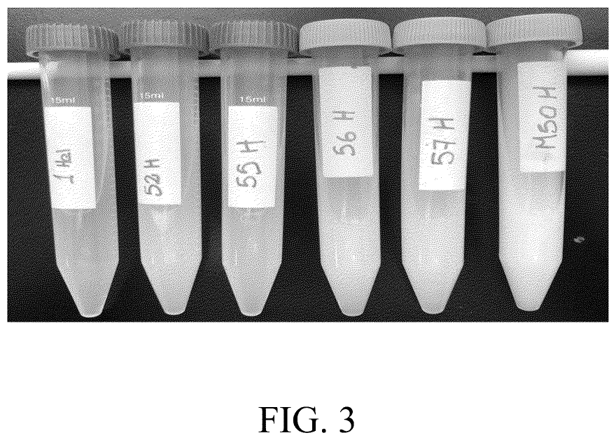

As can be seen, 5 products presented a wetted area greater than or similar to the wetted area of the MSO. Based on the last, this product was performed in a stability test of 1% emulsions in water to verify that their emulsifying properties were preserved. FIG. 3 illustrates the results of these tests. As can be seen, the emulsions formed with these products are stable and homogeneous, similar to the one formed with MSO.

### Conclusion

The following products: AT, EucCy, EucA, EucOA, and OALi are capable of replacing MSO in adjuvant formulations used in agriculture.

### Silwet 641 Adjuvant Replacement

### Laboratory Tests

Several solvents were mixed with humectant MSO, to evaluate their miscibility. The composition of these solvents is as follows: CouT: Coumarin (20-50%), Thymol (50-80%); EucGe: Eucalyptol (30-70%), Geraniol (30-70%); GeLi: Geraniol (30-80%), Limonene (30-70%); EucB: Eucalyptol (20-80%), Borneol (25-75%); ACy: Camphor (30-70%), Cymene (30-70%).

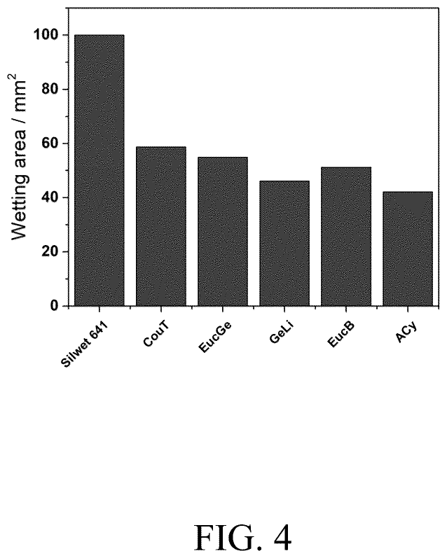

The selection criteria were the miscibility of the proposed solvents with MSO after 1 minute of shaking. The tests were performed in a mixture of 20% Bioeutectics solvent and 80% MSO. In this sense, 86 solvents from our portfolio were tested, of which 56 formed stable mixtures with the humectant, for these products wetting was tested. FIG. 4 shows the wetting area for the selected products.

As can be seen, there are no areas higher than the area for Silwet 641. However, several solvents have 50% of the wetting area, which suggests that they could be improved with optimization.

### Conclusion

The products: CouT, EucGe, GeLi, EucB, and ACy are capable of replacing Silwet 641 in adjuvant formulations used in agriculture.

### N-Methyl Pyrrolidone Replacement

N-methyl pyrrolidone (NMP) is a polar aprotic solvent used in various industries and applications, such as petrochemical, agrochemical, coating, electronic cleaning, and industrial/domestic cleaning. NMP is used in resins, metal-coated plastics, the surface treatment of textiles, or as a paint stripper. In this way, there is a huge demand for this solvent. The NMP global market was calculated to total US$950 million in 2022. It is expected that the market will reach US$1 billion in 2023 and grow at a CAGR of 6.8% from 2023 to 2033. In this context, the demand for this solvent is guaranteed.

However, NMP is known to be quite toxic with several acute effects. NMP can affect the nervous and reproductive system, liver, and kidneys and can cause cancer. Based on this, there is a compelling need to replace this solvent with some safer ones. Bioeutectics offers nature-born solvents derived from renewable resources manufactured by greener and more efficient processes that provide high-performance, natural, and sustainable solvents such as formulating ingredients, carrier solvents, cleaning agents, agrochemical formulation, and antimicrobial agents. Our products are based primarily on a blend of bio-based ingredients like natural organic acids, amino acids, alcohols, sugars, etc. With our technology, liquids can be obtained even if all the compounds are individually solid.

### Laboratory Tests

Initially, products from our portfolio were selected, each one from a different natural family. Taking into account the polarity of 0.45, 0.34, and 0.23 for N-methyl pyrrolidone, acetone, and xylene respectively, Bioeutectics solvents were selected to evaluate all the range of polarities. The composition of these solvents is as follows: TL: Lactic Acid (20-80%), Thymol (20-80%); BT: Borneol (15-40%) and Thymol (60-90%); MOA: Menthol (10-70%) and Oleic Acid (30-90%); MAcA: Menthol (20-90%), Acetic Acid (10-80%); LEuc: Lactic Acid (30-90%), Eucalyptol (30-90%), LC: Lactic Acid (40-80%), Citric Acid (30-60%); OALi: Oleic Acid (30-80%), Limonene (20-90%); LiT: Limonene (30-70%), Thymol (40-70%); GeLi: Geraniol (30-80%), Limonene (30-70%); PiT: Pinene (30-80%), Thymol (40-70%).

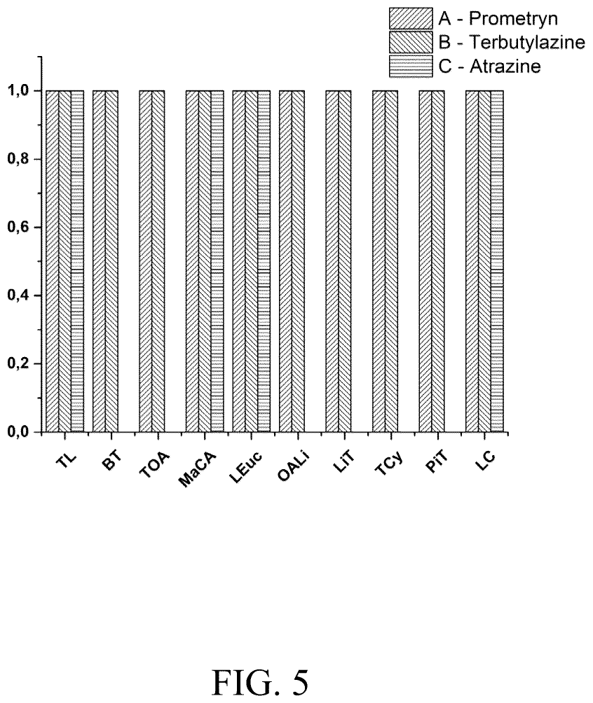

Solubility test (for common agro actives) was carried out at the working concentration of 14% w/w analyte/solvent, initially in traditional solvents and then in our selected solvents, at 30° C. and assisted by vortex and ultrasound. The results were:
Analyte A—Prometryn: Soluble in all our tested solvents.
Analyte B—Terbutylazine: Soluble in 9 of our tested solvents.
Analyte C—Atrazine: Soluble in 4 of our tested solvents.
   FIG. 5 summarizes the results for these experiments. The results are shown in FIG. 5 where 1, and 0 are taken for analytes that are soluble or insoluble, respectively.

It was observed that the solvents capable of dissolving C also dissolved A and B. Therefore, C was considered the problematic analyte with poor solubility, thus the following assays were performed only with C.

Based on this, solubilization tests were performed at different temperatures for C with the select products.

TABLE 6
Solubility of C, % w/v

| | Temperature | | |
|---|---|---|---|
| Solvent | 30° C. | 50° C. | 70° C. |
| TL | 21.4 | 35.7 | 42.9 |
| MAcA | 14.3 | 21.4 | 28.6 |
| LEuc | 10.0 | 14.3 | 21.4 |
| LC | 14.3 | 21.4 | — |

As can be seen in Table 6, TL is the best choice for C solubilization. However, LC could be a good alternative since it is a hydrophilic product.

### Antifreezing Products

The products of the present invention were also tested for their antifreeze properties. Reactors use heating and cooling jackets to remove the heat produced by an endo/exothermic reaction or to provide the heat necessary for a reaction to take place.

Generally, depending on the operating temperature, some thermal oil or a water/glycol mixture are used for heating and cooling jacketed reactors.

In an embodiment, the present invention focuses on NADES products that will be used in reactors with a cooling jacket because they tend to also be suitable for good flow properties at low temperatures.

The minimum target temperature of the non-freeze property was −20° C.

The main purpose of the experiments conducted here was to replace glycols, such as ethylene glycol or propylene glycol, which is typical used for its anti-freeze properties. The chosen NADES products accept water dilutions which decrease the viscosity of the original product.

### Laboratory Tests

Several eutectic systems were tested. For example, the tested systems included the hydrophilic eutectics systems named LGH: Lactic acid (20-90%), Glucose (10-40%), Water (5-15%), and NaAcGlH: Sodium acetate (10-30%), glycerol (30-80%), and water (10-60%). Preferably, LGH: Lactic acid (57%), Glucose (23%), and water (20%), and NaAcGlH: 12% sodium acetate (12%), Glycerol (65%) and water (23%). (The information on characteristics and curves provided corresponds to these percentages)

The systems were prepared by heating them on a magnetic stirrer at 40° C. for 30 min, with continuous agitation until a clear liquid formed.

### Characterization

NaAcGlH was found to have the following properties.

### Freezing

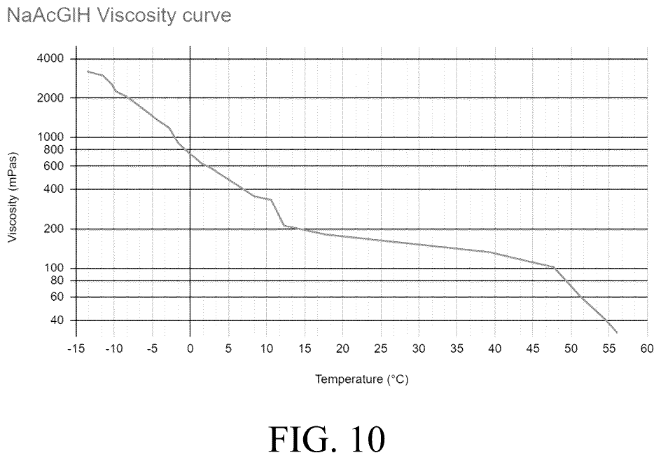

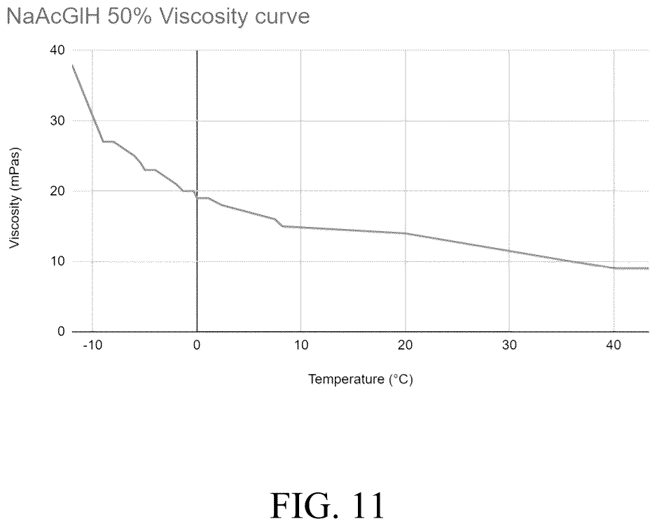

NaAcGlH does not freeze at −80° C. At −20° C. it accepts up to 50% dilution with water without freezing. The viscosity curve of NaAcGlH for the pure solvent system and for 50% water was graphed using a Myr vr3000 viscometer and those curves are shown in FIG. 10 and FIG. 11.

The specific heat was calculated using the Joule calorimeter method and was determined to be 3.0 kJ/kg° C. The specific heat test of the product was carried out with a Joule calorimeter, which is insulated and has a resistance that heats the fluid to be tested. When heating began, the temperature was recorded every 60 seconds and then the heat absorbed per unit of time was determined taking into account the power delivered by the calorimeter. With these data, the value of the specific heat of the solvent was obtained.

The viscosity at 25° C. was determined to be 210 m·Pas (millipascal second). The viscosity at 25° C., dilution of 50% water was determined to be 17 m·Pas. The boiling point was determined to be 130° C. The density was determined to be 1.267 g/ml and conductivity was out of range so it was deduced to be greater than +2000 microSiemens. The pH was 8.5 and the Brix (dissolved solute in a solvent) was 61.1°.

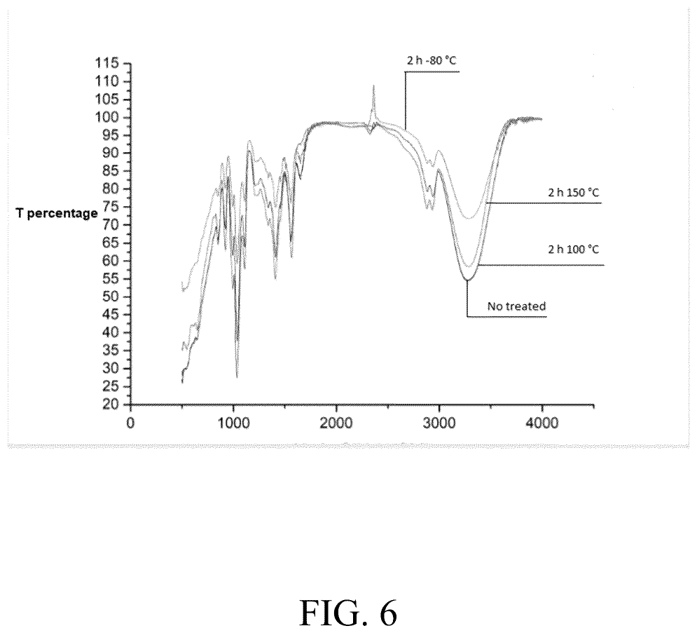

FIG. 6 shows IR spectra corresponding to the untreated product, after 2 h at 150° C., after 2 h at 100° C. and after being subjected to temperatures close to −80° C. It can be seen that the product is thermally stable at these temperature changes, as the spectra do not change appreciably at these vastly different temperatures.

The tests were carried out on an IR equipment Fourier-transformed infrared spectroscopy (FTIR) with Single Attenuated Total Reflectance accessory (QATR-S) (IRSpirit, Shimadzu Corporation, Japan)

The corrosion analysis of the product for different metals was also carried out, the results are detailed in Table 7.

The corrosion analysis was carried out by the Lenor SRL laboratory (Argentina) using the following equipment: Thermohygrometer Testo 608-H1 and Sterilization oven FANEM 515

TABLE 7

| Material | Grading | Designation | Description |
|---|---|---|---|
| Aluminum | 1 | Slight tarnish | Almost same a |
| | | | polished sample |
| Steel | 1 | Slight tarnish | Almost same a |
| | | | polished sample |
| Copper | 1 | Slight tarnish | Light orange, almost |
| | | | same a polished |
| | | | sample |

LGH was found to have the following properties.

### Freezing

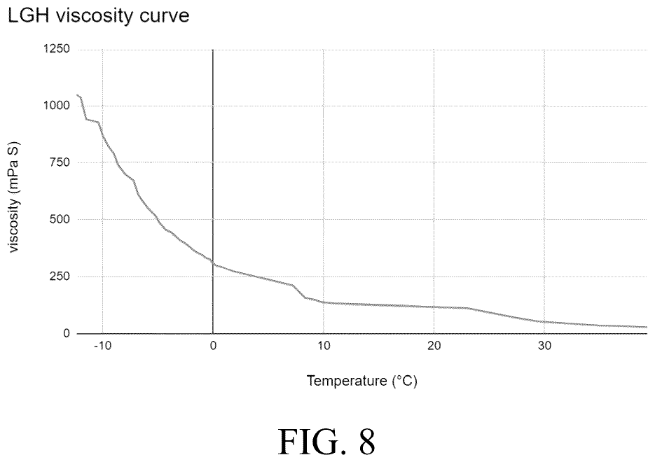

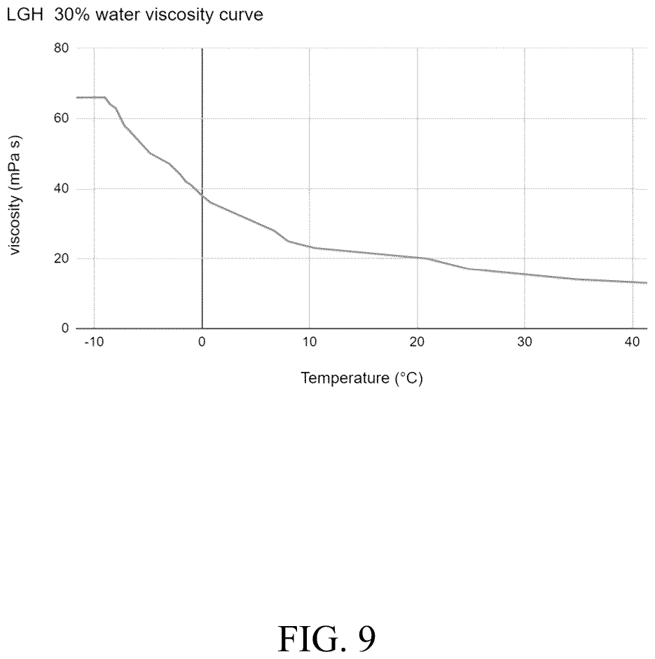

LGH does not freeze at −80° C. At −20° C. it accepts up to 30% dilution water without freezing. The viscosity curve of pure LGH is shown in FIG. 8 and the viscosity curve of LGH containing 30% water is shown in FIG. 9.

The specific heat was calculated using the Joule calorimeter method to be 2.78 kJ/kg° C. and was calculated as described above. Viscosity at 25° C. was determined to be 44 mPa·s and viscosity at 25° C. with 30% water was determined to be 17 mPa·s. The density was determined to be 1.206 g/mL and electrical conductivity was determined to be 370 μS/cm.

The pH was 1.07 and the brix was determined to be 51.8.

### Conclusions Regarding the Anti-Freeze Properties of the Eutectic Products of the Present Invention

The products tested herein can be used in the application of pure cooling jackets and in the proposed dilutions for temperatures up to −20° C. Future experiments will be conducted to more precisely evaluate the ability of the solvents tested herein and other eutectic solvents for their ability to be used for even lower temperatures. The results of the experiments conducted herein suggest that for high temperatures, both of the tested systems should not be subjected to temperatures higher than 120° C. pure or 100° C. after dilution with water.

In an alternative embodiment, the eutectic solvent systems can be added to a glycol such as ethylene glycol or propylene glycol.

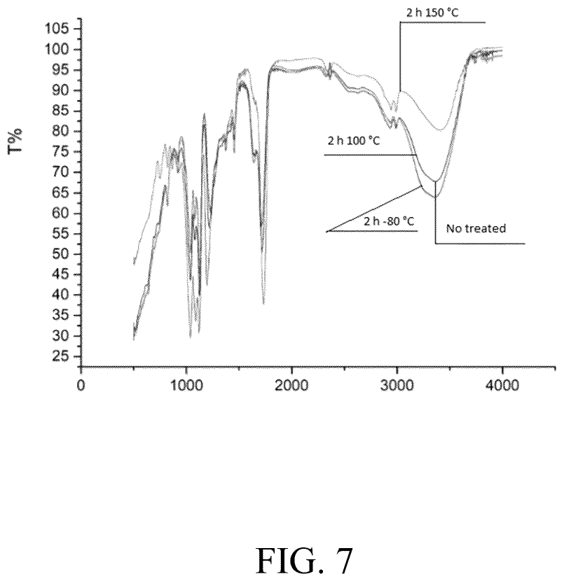

FIG. 7 shows IR spectra of LGH corresponding to the untreated product, after 2 h at 150° C., after 2 h at 100° C. and after being subjected to temperatures close to −80° C. It can be seen that the product is thermally stable only up to 100° C., and not at higher temperatures.

The tests were carried out on a Fourier-transformed infrared spectroscopy (FTIR) instrument with Single Attenuated Total Reflectance accessory (QATR-S) (IRSpirit, Shimadzu Corporation, Japan) B

The corrosion analysis of the product for different metals was also carried out, the results are detailed in Table 8.

The corrosion analysis was carried out by the Lenor SRL laboratory (Argentina) using the following equipment: Thermohygrometer Testo 608-H1 and Sterilization oven FANEM 515.

TABLE 8

| Material | Grading | Designation | Description |
|---|---|---|---|
| Aluminum | 1 | Slight tarnish | Almost same a |
| | | | polished sample |
| Steel | 1 | Slight tarnish | Almost same a |
| | | | polished sample |
| Copper | 1 | Slight tarnish | Light orange, almost |
| | | | same a polished |
| | | | sample |

### Antimicrobial Agents

The present eutectic mixtures were evaluated for their antimicrobial preservation efficacy capacity by Preservative Efficacy Testing (PET), also known as Challenge Testing of selected eutectic systems.

### Product Preparation

The products were prepared as described elsewhere herein. The eutectic systems were prepared by heating the eutectic mixtures on a magnetic stirrer with continuous agitation until a clear liquid formed. The temperature and time were optimized for each product that was prepared.

Both hydrophilic and hydrophobic systems were prepared as above. The hydrophilic systems included LGH: Lactic acid (20-90%), Glucose (10-40%), Water (5-15%), CSH: Citric acid (15-50%), Sorbitol (15-60%), and Water (10-60%), UGl: comprised of urea (and glycerol in a molar ratio of 1:2, UL: urea (20-40%) and lactic (60-80%), TAGl: tartaric acid (10-35%) and glycerol (5-30%) ArgGl: L-arginine (10-45%) and Glycerol (50-90%), BeL: Betaine (10-30%) and lactic acid (20-40%) CGlH: Citric Acid (25-90%), Glycerol (5-70%), Water (1-25%), ArgLH: L-arginine (10-30%) lactic acid (20-50%), and water (5-25%), BeUH: Betaine (10-40%), Urea (10-25%), and Water (10-40%), BeGH: Betaine (10-25%), Glucose (5-30%), and Water (20-60%), LSH: Lactic acid (10-60%), Sorbitol (5-30%), and Water (5-25%), MAL: Malic acid (10-30%), and Lactic acid (30-60%), and LGlMA: Lactic acid (5-30%), Glycerol (5-25%), and Malic acid (10-30%).

The hydrophobic systems included AT: Camphor (30-60%), Thymol (40-70%), AM: Camphor (25-45%), Menthol (55-75%), and ML: Menthol (25-65%), Lactic Acid (35-75%), and GeOA: Geraniol (10-40%), Oleic Acid (30-90%), MOA: Menthol (10-70%), Oleic Acid (30-90%), GeM: Geraniol (10-40%), and Menthol (25-70%), EucA: Eucalyptol (10-60%) and Camphor (5-30%), and OALuA: Oleic acid (20-70%) and lauric acid (5-25%).

Moreover, the LGH system was diluted with water at 50%, 25%, 10%, 3%, and 0.3%. These systems were also studied for their anti-microbial properties.

### Preservation Efficiency

The present eutectic mixtures were tested by Challenge test to ascertain their putative anti-microbial properties. Challenge Test is a procedure in which a product is challenged by exposure to specified types of bacteria and fungi to determine whether it is adequately preserved. Test organisms should be representative of those likely to occur as contaminants during use and should consist of Gram-positive and Gram-negative bacteria, mold, and yeast. The microorganisms are inoculated in product samples, and aliquots are removed at appropriate intervals to determine survivors.

An initial level of contamination that was tested was 30×106 UFC/mL.
The following bacterial, yeast, and mold microbes were tested.
Gram-positive bacteria: Staphylococcus aureus
Gram-negative bacteria: Escherichia coli, Pseudomonas aeruginosa
Yeast: Candida sp
Mold: Aspergillus niger

The inoculated products were held at room temperature and were evaluated at specific intervals over a 28-day period. After 48 hours of incubation, surviving microorganisms were counted, and the reduction of each microorganism at each interval was reported.

For all systems tested, there was an absence after 48 hours of the Escherichia coli, Staphylococcus aureus, Pseudomonas aeruginosa, Candida sp, and Aspergillus niger. The testing was done by a lab entitled Labaratorio Bado.

The results obtained from Preservative Efficacy Testing are crucial for ensuring that the eutectic mixture meets the required standards for microbial protection in various cosmetic and personal care products. It helps determine if the formulation is capable of maintaining its microbial stability and safety throughout its intended shelf life.

Thus, each of the tested systems demonstrated efficacy as an antimicrobial system. On the other hand, the eutectic systems that are tested herein for their antimicrobial properties can be replaced or supplemented by one or more preservatives commonly used in cosmetics and pharmaceutical products like methylparaben, ethylparaben, propylparaben, butylparaben, and benzylic alcohol, among others.

It is contemplated that the presently tested systems can further be combined with other known antimicrobial products to give enhanced antimicrobial effects. In an embodiment, the presently tested systems may be combined with one or more of an antibiotic product selected from the group consisting of penicillin, amoxicillin, doxycycline, cephalexin, ciprofloxacin, clindamycin, metronidazole, azithromycin, sulfamethoxazole, and trimethoprim.

### Personal Care Products

The objective of the experiments performed herein was to evaluate changes in organoleptic characteristics (such as smell, color, texture, others, and combinations thereof) in emulsions after the addition of a NADES as an additive. An additional aim was to analyze changes in some physicochemical characteristics in the base cream and to observe variations according to the NADES's characteristics. One more aim was to validate the possibility of using NADES as additives in base cream(s) in different proportions to attain different properties. The addition of the NADES was expected to provide superior benefits relative to preservative capacity using the product that is added to the NADES alone.

### Laboratory Tests

The following lab tests were performed. The NADES products were prepared so that several different eutectic systems were tested. Both hydrophilic and hydrophobic eutectic systems were prepared. The hydrophilic eutectics systems included LGH: Lactic acid (20-90%), Glucose (10-40%), Water (5-15%), UGIH: Urea (10-55%), Glycerol (15-80%), Water (0-35%), UL: urea (20-40%) and lactic (60-80%), BeL: Betaine (10-30%) and lactic acid (20-40%), ArgCH: L-arginine (5-25%), Citric acid (10-30%) and water (15-40%), ArgLH: L-arginine (10-30%) lactic acid (20-50%), and water (5-25%), BeUH: Betaine (10-40%), Urea (10-25%), and Water (10-40%), BeSH: Betaine (15-35%), Sorbitol (20-40%), and water (5-20%), BeGlG: Betaine (5-25%), Glycerol (5-40%), and Glucose (5-30%), BcC: Coconut betaine (20-60%) and Citric acid (10-30%), LSH: Lactic acid (10-60%), Sorbitol (5-30%), and Water (5-25%). The hydrophobic eutectic systems included MOA: Menthol (10-70%), Oleic Acid (30-90%), and OALuA: Oleic acid (20-70%) and lauric acid (5-25%).

The various NADES systems were prepared by heating and stirring the compounds in the system on a magnetic stirrer at 40° C. for 45 min, with continuous agitation until a clear liquid formed.

### Incorporation of NADES as Additives in Base Cream

An experiment was performed to evaluate various NADES systems as additives. They were incorporated into a basic cosmetical cream at concentrations of 1% and 15%, except MOA, which was only used at a concentration of 1%. These proportions were planned according to and deduced from concentrations used in emulsion formulations while considering their application as additives (generally, additives are kept at amounts below about 10%).

### Results

The following observations were noted:

For the NADES solvent system LGH, additives were well incorporated in both proportions and viscosity decreased at 15%. No changes in smell and/or color were observed. One month after the beginning of the trial, at 15% the cream presented a bit of an acidic smell indicating that the formulation may have changed. However, the consistency of the formulation was maintained. No observable changes were seen at 1% concentration.

For the NADES solvent system UL, additives were well incorporated in both proportions and viscosity decreased at the 15% level. No changes in smell and color were observed. One month after the beginning of the trial, the cream presented a lactic acid smell at 15%. However, the consistency of the formulation was maintained. There were no observable changes at the 1% concentration level.

For the NADES solvent system, BeL, additives were well incorporated in both proportions and viscosity decreased at the 15% level. A color change was noted in the formulation to an amber color that was more similar to the color of the NADES system. One month after the beginning of the trial, the cream presented a lactic acid smell at the 15% level. The consistency of the formulation was maintained. No observable changes occurred at the 1% concentration level.

For the NADES solvent system, ArgLH, it was difficult to incorporate the additives because of the high viscosity of the NADES. There were no observable changes in smell and color. Moreover, there were no observable changes after one month from the beginning of the trial.

For the NADES solvent system, ArgCH, it was difficult to incorporate the additives because the viscosity at the 15% concentration level of the NADES increased because of the high viscosity of the NADES. No changes in smell and color were observed. No observable changes were noted one month after the beginning of the trial.

For the NADES solvent system, BeUH, it was difficult to incorporate the additives. There was a change in color at the 15% concentration level and the formulation changed to a color that was similar to the color of the NADES. No observable changes in smell occurred. Moreover, there were no observable changes noted one month after the beginning of the trial.

For the NADES solvent system, MOA, no changes were observed with the addition of the NADES. However, one month after the beginning of the trial, a menthol-like smell was detected.

### pH Measurements

The pH remained relatively stable during the trial. The pH level of the respective formulations are shown in Table 9.

TABLE 9

| | pH of the cream + | pH of the cream + |
|---|---|---|
| NADES | NADES at 1% | NADES at 15% |
| LGH | 2.56 | 2.21 |
| UL | 2.56 | 2.09 |
| BeL | 2.95 | 2.81 |
| ArgLH | 3.72 | 3.34 |
| BeCH | 6.58 | 7.56 |
| BeUH | 6.42 | 7.57 |
| MOA | 5.84 | 5.80 |

### CONCLUSIONS

The NADES systems all appear to be useful for the purpose of being added to personal care products. In this regard, all additives were stable at the concentrations tested and they demonstrated stability over time. All of the NADES systems of the present invention demonstrated preservation efficacy, use as preservative systems, and use as additives for functional foods, cosmetics, and personal care products.

Of note, LGH maintained its efficiency at all dilutions studied.

In an embodiment, the present invention relates to NADES systems that comprise eutectic solvents that can be added to any of a plurality of products. In a variation, the addition of the NADES system to the plurality of products makes the products better than the products in the absence of the NADES system. In a variation, the eutectic solvents comprise more than one of the following solvents: menthol, lactic acid, thymol, oleic acid, urea, glucose, water, L-arginine, tartaric acid, citric acid, sodium acetate, glycerol, borneol, sorbitol, betaine, or camphor. In a variation, the particular solvent system mix is as disclosed herein in the relative amounts as disclosed herein. In a variation, the product relates to one or more of an adjuvants, an anti-freeze product, an antimicrobial product, or a personal care product. In a variation, the personal care product may be a cream (such as moisturizing cream), a mouthwash, a soap, a cologne, a moisturizer, an antiperspirant, hair removal products, body butter, cleanser, conditioner, eye cream, sunscreen, suntan lotion, hand sanitizer, exfoliator, shampoo, toothpaste, skin care products, or other personal care products. In a variation, the antimicrobial products include but are not limited to disinfectants, sterilants, fungicides, or antibacterial products. The antibacterial products may be incorporated into the personal care products disclosed herein. In a variation, they may be incorporated into cleaning products, in soaps and/or detergents, in hand lotions, in disinfectants, in window cleaners, in cleaning cloths, in surface sprays, in mouthwashes, in toothpastes, in plastic wrap, or in textiles and carpeting.

In an embodiment, the NADES system may be added to a product that is used to act as a herbicide, a fungicide, or a bactericide or against plants, fungi, or bacteria.

In an embodiment, the present invention relates to a composition that comprises the NADES systems as disclosed herein along with a product. The product can be any of the products disclosed herein.

In a variation, the NADES system can be added to antifreeze compounds including but not limited to mono ethylene glycol, mono propylene glycol, methanol, propylene glycol methyl ether, various organic acids such as 2-ethylhexanoic acid, or other antifreeze compounds. In a composition that comprises a NADES and an antifreeze compound, the composition may also optionally contain additives such as sodium silicate, disodium phosphate, sodium molybdate, sodium borate, denatonium benzoate, and/or dextrin or other additives. In a variation, the antifreeze may actually be a mix of two or more of the above listed compounds (or of water). Thus, the product may not just be a pure compound but may actually be a mix of compounds.

In an embodiment, the NADES system and the eutectic solvent mixture may be added to an adjuvant. An adjuvant typically is a compound that can be added to a medicine, vaccine, or drug, that increases the efficacy of that medicine vaccine or drug. Thus, in an embodiment, the present invention relates to a compositional mix that comprises a compound with pharmacological properties, an adjuvant, and a NADES system of eutectic solvents. In a variation, the eutectic solvents may enhance solubility of the adjuvant and or the compound with pharmacological properties allowing either greater doses to be administered or increasing the efficacy of the dose administered. In a variation, the adjuvant may be enhancing the delivery system or act as an immune potentiator (stimulating the immune system).

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. A NADES system that comprises a eutectic solvent system that is useful as an additive to an adjuvant, an anti-freeze product, an antimicrobial product, or as a personal care product.

2. The NADES system of claim 1, wherein the eutectic solvent system enhances qualities of a product that is an adjuvant, an anti-freeze product, an antimicrobial product, or a personal care product.

3. The NADES system of claim 1, wherein the eutectic solvent system comprises one or more of the following compounds: menthol, lactic acid, thymol, oleic acid, urea, glucose, water, L-arginine, tartaric acid, citric acid, sodium acetate, glycerol, borneol, sorbitol, betaine, or camphor.

4. The NADES system of claim 1, wherein the eutectic solvent system is for an anti-freeze product, and the eutectic solvent comprises one or more of lactic acid, glucose, sodium acetate, glycerol and water.

5. The NADES system of claim 1, wherein the eutectic solvent system is for an antimicrobial product, and the eutectic solvent comprises one or more of lactic acid, glucose, water, citric acid, sorbitol, urea, glycerol, tartaric acid, L-arginine, camphor, and menthol.

6. The NADES system of claim 1, wherein the eutectic solvent system is for an adjuvant and the eutectic solvent system comprises one or more of urea, lactic acid, menthol thymol, oleic acid and borneol.

7. The NADES system of claim 1, wherein the eutectic solvent system is for a personal care product and the eutectic solvent system comprises one or more of lactic acid, glucose, water, urea, betaine, L-arginine, citric acid, water, menthol, and oleic acid.

8. The NADES system of claim 7, wherein the eutectic solvent system is added to a cosmetical cream product.

9. A composition comprising a NADES and a compound that is used as an adjuvant, an anti-freeze product, an antimicrobial product, or a personal care product.

10. The composition of claim 9, wherein the compound is an adjuvant that is Triton X-100.

11. The composition of claim 9, wherein the compound and the NADES is effective against one or more of Escherichia coli, Staphylococcus aureus, Pseudomonas aeruginosa, Candida sp, and/or Aspergillus niger.

12. The composition of claim 9, wherein the compound is an antifreeze product, which is a glycol.

13. The composition of claim 12, wherein the glycol is ethylene glycol or propylene glycol.

14. The composition of claim 9, wherein the compound is a personal care product and the personal care product is a cosmetical cream.

15. The composition of claim 11, wherein the composition is effective at treating a bacterial infection caused by Staphylococcus aureus, Escherichia coli, or Pseudomonas aeruginosa.

16. A method of treating an individual that suffers from a bacterial infection caused by Staphylococcus aureus, Escherichia coli, or Pseudomonas aeruginosa, said method comprising administering to said individual a eutectic solvent system and an antimicrobial compound or composition.

17. The method of claim 16, wherein the eutectic solvent system comprises two or more of lactic acid, glucose, water, citric acid, sorbitol, urea, glycerol, tartaric acid, L-arginine, camphor, or menthol.

18. The method of claim 17, wherein the eutectic solvent system comprises a) lactic acid, glucose, and water, b) citric acid, sorbitol, and water c) urea and glycerol d) urea and lactic acid, e) tartaric acid and glycerol f) L-arginine and glycerol, g) camphor and thymol h) camphor and menthol or i) menthol and lactic acid.

19. The composition of claim 9, wherein the NADES is one or more compounds selected from the group consisting of menthol, lactic acid, thymol, oleic acid, urea, glucose, water, L-arginine, tartaric acid, citric acid, sodium acetate, glycerol, borneol, sorbitol, betaine, and camphor.
